(19) United States
(12) Patent Application Publication
(10) Pub. No.: US 2025/0390734 A1
(43) Pub. Date: Dec. 25, 2025

(54) Method of Identifying Cardiovascular Parameters and Disease

(71) Applicant: Anika Pallapothu, Cupertino, CA (US)

(72) Inventor: Anika Pallapothu, Cupertino, CA (US)

(21) Appl. No.: 18/752,529

(22) Filed: Jun. 24, 2024

(51) Int. Cl. G06N 3/08; A61B 5/00; G06T 7/00

(52) U.S. Cl. CPC G06N 3/08; A61B 5/0044; G06T 7/0012; G06T 2207/10088; G06T 2207/30048

(57) ABSTRACT

A robust deep learning artificial intelligence or AI based automated approach to cardiac MRI is provided that can be used to diagnose a patient as being normal, having systolic heart failure with infarction, dilated cardiomyopathy, hypertrophic cardiomyopathy or abnormal right ventricle, and/or other diagnoses with the input of a cardiac MRI scan. Along with the diagnosis, a detailed quantitative analysis of cardiac parameters like volumes of left and right ventricles and myocardium at systole and diastole phases, along with myocardial wall thickness and also the ejection fraction of the left and right ventricles area are provided.

### FIELD OF THE INVENTION

The invention relates to methods for identifying cardiovascular parameters and disease.

### BACKGROUND OF THE INVENTION

Cardiovascular diseases (CVD) are leading causes of diseases in the world. Heart attack and stroke are the most common fatalities related to CVDs so a timely diagnosis is crucial to its prevention. Cardiac MRI is often used for diagnosis of cardiovascular diseases. It helps in giving a detailed and quantitative analysis of the parameters associated with heart's anatomy. However, there are problems with speed, access, accuracy and consistency of diagnosis of CVDs using cardiac MRI and better solutions are needed.

### SUMMARY OF THE INVENTION

Certain embodiments of this invention comprise a robust deep learning artificial intelligence or AI based automated approach that could diagnose a patient as being normal, having systolic heart failure with infarction, dilated cardiomyopathy, hypertrophic cardiomyopathy or abnormal right ventricle, with the input of a cardiac MRI scan. Along with the diagnosis, a detailed quantitative analysis of cardiac parameters like volumes of left and right ventricles and myocardium at systole and diastole phases, along with myocardial wall thickness and also the ejection fraction of the left and right ventricles is provided.

As soon as the patient is scanned, the AI model of this invention picks up and processes the data and presents an automated report in the desired format with a detailed quantitative analysis. These AI models of this invention give an accuracy of at least 95% on diagnosis and at least 92% on quantitative analysis when checked by hand.

In the past, manual delineation of cardiac parameters and performing quantitative analysis was tedious and time consuming, especially with the introduction of intra and inter-rater variability. Such an approach would be difficult to follow in large hospitals with high footprint. With the AI assisted models of this invention, the report can be generated within seconds that can be used by doctors as a cross-reference for further diagnosis of the patients. This easily reduces the time spent on analyzing the raw data and gives meaningful insights that could be missed when tried to evaluate manually in a larger footprint scenario.

The AI model used should be rigorously tested across different centers with images acquired across a wide range of scanners with different acquisition protocols. In some embodiments, the AI model is focused to classify cardiac MRI into five classes. In preferred embodiments, more classes are added.

In certain preferred embodiments of this invention, methods of processing and analyzing a first set of MRI images of cardiac tissue and outputting a report are provided. The methods comprise: (a) acquiring a second set of MRI images of cardiac tissue that have been previously rated for known cardiac parameters, disease states, scanner type, and acquisition protocol; (b) training deep learning artificial intelligence on the second set of MRI images of cardiac tissues that have been previously rated in order to obtain a trained deep learning artificial intelligence; (c) acquiring the first set of MRI images of cardiac tissues; (d). applying the trained deep learning artificial intelligence to the first set of MRI images of cardiac tissues; and (e) outputting the report from the trained deep learning artificial intelligence on the first set of MRI images of cardiac tissues comprising cardiac parameters, disease states, scanner type, and acquisition protocol.

Additional steps can be added to these preferred embodiments, including: a further step of applying the trained deep learning artificial intelligence to reconstruct the first set of MRI images in order to improve them and remove artefacts: a further step of applying the trained deep learning artificial intelligence to registration of landmarks of the first set of MRI images to provide for motion compensation for temporal image sequences; and/or a further step of applying the trained deep learning artificial intelligence to generate reports concerning the first set of MRI images to provide for accelerated report generation using standardized and uniform forms.

In other preferred embodiments of this invention methods of analyzing a first set of MRI images of cardiac tissue and outputting a report are provided. The methods comprise: (a) submitting the first set of MRI images to a trained deep learning artificial intelligence, the trained deep learning artificial intelligence having been trained with a second set of MRI images of cardiac tissue that have been previously rated for known cardiac parameters, disease states, scanner type, and acquisition protocol; and (b) outputting the report from the trained deep learning artificial intelligence on the first set of MRI images comprising cardiac parameters, disease states, scanner type, and acquisition protocol.

Additional steps can be added to these preferred embodiments also, including: a further step of applying the trained deep learning artificial intelligence to reconstruct the first set of MRI images in order to improve them and remove artefacts; a further step of applying the trained deep learning artificial intelligence to registration of landmarks of the first set of MRI images to provide for motion compensation for temporal image sequences; and/or a further step of applying the trained deep learning artificial intelligence to generate reports concerning the first set of MRI images to provide for accelerated report generation using standardized and uniform forms.

In still other preferred embodiments of this invention, methods of generating a report on a first set of MRI images of cardiac tissue are provide. The methods comprise: (a) submitting the first set of MRI images to a trained deep learning artificial intelligence, the trained deep learning artificial intelligence having been trained with a second set of MRI images of cardiac tissue that have been previously rated for known cardiac parameters, disease states, scanner type, and acquisition protocol; and (b) outputting the report from the trained deep learning artificial intelligence on the first set of MRI images comprising cardiac parameters, disease states, scanner type, and acquisition protocol.

Additional steps can be added to these preferred embodiments also, including: a further step of applying the trained deep learning artificial intelligence to reconstruct the first set of MRI images in order to improve them and remove artefacts; a further step of applying the trained deep learning artificial intelligence to registration of landmarks of the first set of MRI images to provide for motion compensation for temporal image sequences; and/or a further step of applying the trained deep learning artificial intelligence to generate reports concerning the first set of MRI images to provide for accelerated report generation using standardized and uniform forms.

Advantages of the embodiments of this invention are described and apparent throughout this specification. For example, certain embodiments automate the examination of MRI images for cardiac data at a higher speed and useful accuracy. Further advantages will be apparent to a person of skill in the art applying the embodiments of the invention.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### DETAILED DESCRIPTION OF THE INVENTION

The AI model of this invention is trained on MRI images with known parameters and disease states from one or more individual raters (e.g., radiologists). Parameters that should be identified with the training set include the particular scanner, acquisition protocol, and type of disease or other data of interest. Additional factors can be the person who was the rater of the training images.

In one set of embodiments, a tagged set of MRI images is created by a rater. In certain of these embodiments, the tagged set of MRI images is used to create a set of synthetic data and images for training the AI model.

Preferred embodiments of this invention use deep learning, which is a subset of machine learning that is based on a neural network structure loosely inspired by the human brain. Such structures learn discriminative features from data automatically, giving them the ability to approximate very complex nonlinear relationships. While most earlier AI methods have led to applications with subhuman performance, deep learning algorithms are able to match and even surpass humans in task-specific applications. Deep learning is applied to both detection and segmentation tasks in these embodiments.

Certain embodiments of this invention can aid in accomplishing particular tasks in cardiac MRI. One such task is reconstruction of images, using deep learning methods for suppressing artefacts and improving overall quality. Deep learning in learning reconstruction transformations for various MRI acquisition strategies is achieved in some embodiments by treating the reconstruction process as a supervised learning task where a mapping between the scanner sensors and resultant images is derived. Other efforts use the AI to correct for artefacts.

Another task to apply this invention to is registration. This process may be based on predefined similarity criteria such as landmark and edge-based measures. Deep learning methods of this invention may handle complex tissue deformations and provide better motion compensation for temporal image sequences.

Radiology reports often lie at the intersection of radiology and specific practices. However, the generation of these textual reports can be a laborious and routine time-consuming task. AI-run, automatic, report-generation tools of this invention may provide for a faster, more thorough, more uniform, report, which may also use more standardized terminology.

In certain embodiments, the output from the AI can be focused to a particular practice or need (e.g., disease of interest, type of patient (e.g., geriatric, pediatric, athlete)), with findings such as the cardiac tissue being normal, having systolic heart failure with infarction, dilated cardiomyopathy, hypertrophic cardiomyopathy or abnormal right ventricle, with the input of a cardiac MRI scan to the trained AI. Along with the diagnosis, a detailed quantitative analysis of cardiac parameters can be provided. This can include volumes of left and right ventricles and myocardium at systole and diastole phases, along with myocardial wall thickness and also the ejection fraction of the left and right ventricles. One of skill in the art will recognize that other aspects of the cardiac tissue can be evaluated and measured.

The reports output by the AI model preferably mimic the report used by the specific location or practice so the information is readily available. The report should clearly identify the origin of the information provided, including the AI model.

### Other Embodiments

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

What is claimed is:

1. A method of processing and analyzing a first set of MRI images of cardiac tissue and outputting a report, the method comprising:
a. acquiring a second set of MRI images of cardiac tissue that have been previously rated for known cardiac parameters, disease states, scanner type, and acquisition protocol;
b. training deep learning artificial intelligence on the second set of MRI images of cardiac tissues that have been previously rated in order to obtain a trained deep learning artificial intelligence;
c. acquiring the first set of MRI images of cardiac tissues;
d. applying the trained deep learning artificial intelligence to the first set of MRI images of cardiac tissues; and
e. outputting the report from the trained deep learning artificial intelligence on the first set of MRI images of cardiac tissues comprising cardiac parameters, disease states, scanner type, and acquisition protocol.

2. The method of claim 1, with a further step of applying the trained deep learning artificial intelligence to reconstruct the first set of MRI images in order to improve them and remove artefacts.

3. The method of claim 1, with a further step of applying the trained deep learning artificial intelligence to registration of landmarks of the first set of MRI images to provide for motion compensation for temporal image sequences.

4. The method of claim 1, with a further step of applying the trained deep learning artificial intelligence to generate reports concerning the first set of MRI images to provide for accelerated report generation using standardized and uniform forms.

5. A method of analyzing a first set of MRI images of cardiac tissue and outputting a report, the method comprising:
a. submitting the first set of MRI images to a trained deep learning artificial intelligence, the trained deep learning artificial intelligence having been trained with a second set of MRI images of cardiac tissue that have been previously rated for known cardiac parameters, disease states, scanner type, and acquisition protocol; and
b. outputting the report from the trained deep learning artificial intelligence on the first set of MRI images comprising cardiac parameters, disease states, scanner type, and acquisition protocol.

6. The method of claim 5, with a further step of applying the trained deep learning artificial intelligence to reconstruct the first set of MRI images in order to improve them and remove artefacts.

7. The method of claim 5, with a further step of applying the trained deep learning artificial intelligence to registration of landmarks of the first set of MRI images to provide for motion compensation for temporal image sequences.

8. The method of claim 5, with a further step of applying the trained deep learning artificial intelligence to generate reports concerning the first set of MRI images to provide for accelerated report generation using standardized and uniform forms.

9. A method of generating a report on a first set of MRI images of cardiac tissue, the method comprising:
a. submitting the first set of MRI images to a trained deep learning artificial intelligence, the trained deep learning artificial intelligence having been trained with a second set of MRI images of cardiac tissue that have been previously rated for known cardiac parameters, disease states, scanner type, and acquisition protocol;
b. outputting the report from the trained deep learning artificial intelligence on the first set of MRI images comprising cardiac parameters, disease states, scanner type, and acquisition protocol.

10. The method of claim 9, with a further step of applying the trained deep learning artificial intelligence to reconstruct the first set of MRI images in order to improve them and remove artefacts.

11. The method of claim 9, with a further step of applying the trained deep learning artificial intelligence to registration of landmarks of the first set of MRI images to provide for motion compensation for temporal image sequences.

12. The method of claim 9, with a further step of applying the trained deep learning artificial intelligence to generate reports concerning the first set of MRI images to provide for accelerated report generation using standardized and uniform forms.
